(19) United States
(12) Patent Application Publication
(10) Pub. No.: US 2002/0050080 A1
(43) Pub. Date: May 2, 2002

(54) Orthotic device

(76) Inventor: Phillip J. Vasyli, Kirrawee (AU)

Correspondence Address: KINNEY & LANGE, P.A., THE KINNEY & LANGE BUILDING, 312 SOUTH THIRD STREET, MINNEAPOLIS, MN, 55415-1002, US

(21) Appl. No.: 09/855,206

(22) Filed: May 14, 2001

Related U.S. Application Data
(63) Continuation of application No. 08/963,540, filed on Nov. 3, 1997, now abandoned.
Application No. 08/963,540 is a continuation of application No. 08/090,032, filed on Jul. 9, 1993, now abandoned.
Application No. 08/090,032 is a 371 of international application No. PCT/AU91/00185, filed on Apr. 30, 1991.

(51) Int. Cl. A43B 7/14; A61F 5/14

(52) U.S. Cl. 36/145; 36/154; 36/166; 36/173

(57) ABSTRACT

A contoured, pre-molded and heat moldable orthotic device (1), ideally of about three-quarters of the length of the boot or shoe in which it is to be worn, has a 4° rear foot varus angle or wedge with an integrally-formed heel cup (3), a longitudinally-extending “raise” (4), a metatarsal “raise” (5) and a spongy shock-absorbing insert (6). The device is intended to be purchase “off-the-shelf”, for the relief of generalized and relatively minor common biomechanical foot problems.

### TECHNICAL FIELD

This invention relates to orthotic devices and more particularly to such a device which may be purchased through retail outlets and which may provide the purchaser with an inexpensive product well able to remedy common biomechanical problems relating to the foot.

### BACKGROUND ART

In the specification of Patent Cooperation Treaty application no. PCT/AU90/00543 filed on Nov. 9, 1990 there is described and illustrated an orthotic device able to be molded to a patient's foot, “in-situ” in an article of footwear, to give support to, and to control, the osseous structures of the foot. It is envisaged that such devices would be molded and fitted by a medical practitioner, an orthopaedic surgeon or, at least, a specialist technician. By way of contrast, the device of the present invention is adapted. to be purchased and installed by the layman; as may aptly be said, the device in accordance with application no. PCT/AU90/00543 is a specialized “correcting device” while the present invention is an orthotic device for general use and having generalized correcting qualities.

Other than the above-mentioned moldable, or molded, orthotic devices, so-called “arch-supporters” are known, ranging from simple contoured insoles to costly structures integrally incorporated into made-to-order orthopaedic footwear.

Orthopaedic footwear apart, the known insertable insole devices usually overlie the whole of the upper surface of the liner of the shoe sole, thus requiring the existence of an extensive range of lengths, widths and even shapes—for example, the court-fit shape in women's dress shoes. These known insoles are sold “off the shelf”, as a rule, and provide but indifferent biomechanical control or, if they are fitted to the patient's shoe, the fitting and adjustment time may be quite long. They rarely provide satisfactory biomechanical control for, and control of, the osseous structures of the foot and are often made of unyielding materials such as hard plastic or carbon fibre; on the other hand, many resilient molded insoles merely function as shock absorbers. “Full length” orthotic devices are disclosed in such U.S. patents as U.S. Pat. No. 3,895,405 (EDWARDS); U.S. Pat. No. 3,782,390 (JOHNSON); U.S. Pat. No. 2,760,281 (COSIN) and U.S. Pat. No. 2,409,594 (SHERMAN), while U.S. Pat. Nos. 4,674,204; 4,232,457; 3,825,017 and 4,522,777; to SULLIVAN, MOSHER, SCRIMA and PETERSON respectively disclose various orthotic insoles having spongy or foam layers.

In U.S. Pat. No. 2,401,514 (SCHOLL) the inventive concept is a strip of fabric 15 applied to the underside of a thermoplastic resin arch support to prevent it from sliding and squeaking when worn inside a shoe; U.S. Pat. No. 4,517,981 (SANTOPIETRO) relates to a substantially flat, three-quarter length orthotic device having no longitudinal arch “raise” or metatarsal “raise”. U.S. Pat. No. 4,823,420 (BARTNECK). discloses a contour molded insole, including several layers of material; it is apparently somewhat less than three-quarter length and it is abitrarily cut off in a straight transverse front edge provided with no metatarsal “raise”.

Other specifications of interest are U.S. Pat. No. 2,53,396 (GOTTLIEB); U.S. Pat. No. 3,068,872 (BRODY); 3,121,431 (ROSENHAFT); U.S. Pat. No. 3,309,797 (POATIS); U.S. Pat. No. 4,216,778 (WEISS); U.S. Pat. No. 4,268,980 (GUDAS); U.S. Pat. No. 4,346,525 (LARSEN); U.S. Pat. No. 4,364,188 (TURNER); U.S. Pat. No. 4,463,761 (POIS); U.S. Pat. No. 4,520,581 (IRWIN); U.S. Pat. No. 4,530,173 (GESINSKY); U.S. Pat. No. 4,557,060 (KAWASHIMA); U.S. Pat. No. 4,563,787 (DREW); U.S. Pat. No. 4,674,201 (WEISS); U.S. Pat. No. 4,702,255 (SCHENKI); U.S. Pat. No. 4,756,096 (MAYER); U.S. Pat. No. 4,791,736 (PHILLIPS) and U.S. Pat. No. 4,868,945 (DE VETTIGNIS).

U.S. Pat. No. 3,859,740 (KEMP) discloses a “cushion pad for heel spurs” consisting of three layers and an insert, and U.S. Pat. No. 486,993 (GRUMBINE) described and illustrates, in a second embodiment of the invention, a “rigid unitary contoured supportive plastic shell” including a lateral cut-away portion.

### DISCLOSURE OF INVENTION

It is therefore an object of the present intention to overcome or, at the very least, to mitigate those disadvantages and shortcomings which will be perceived as being inherent in the above and other prior art documents by the provision of a contoured moldable orthotic device adapted to be inserted into an article of footwear so as to overlie at least a part of the upper surface of a sole thereof, to thereby give support to, and to control, the osseous structures of the foot; said orthotic device being formed with an integrally-molded heel cup, a longitudinal arch raise, a varus post angled at about 4° and a metatarsal raise for aligning the heads of the second, third and fourth metatarsals; the said heel cup incorporating a low-density sponge-like, shock-absorbing insert adapted to underlie that area of a patient's foot which is beneath the heel bone, to thereby cushion the foot throughout the heel strike thereof.

Ideally, the shock-absorbing insert includes an integral “shock dot” portion—preferably this insert is formed from 30-50 kg/M³ density polyurethane foam—adapted to cushion that part of a said patient's foot which lies directly beneath a heel spur or calcaneus of the foot; the remainder of the device being formed from ethyl vinyl acetate of 150-350 kg/M³ density, and ideally of 220 kg/M density. Advantageously, the upper surface of the inventive device may well be covered with a fabric-like outer “skin”.

Ideally, the width of the moldable orthotic device is that distance from the lateral aspect of the head of the fifth metatarsal to the longitudinal bisection of the first and second metatarsals; the arrangement being such that the shaft of the first metatarsal is able to plantarflex during the propulsive phase of a said patient's foot. The outer edge of the device may well be laterally cut away, to improve the fitting qualities of the device into the shoe.

The terms “raise”, “plantarflex” and “varus post” are well understood by those familiar with the field of orthotic devices.

### BRIEF DESCRIPTION OF THE DRAWINGS

In order that a better understanding of the present invention may be gained, hereinafter will be described preferred embodiments thereof, by way of example only and with reference to the accompanying drawings in which:

FIG. 1 is a bottom plan view of a device in accordance with the present invention; for a right foot;

FIG. 2 is a top plan view thereof;

FIG. 3 shows the right or outer side of the device;

FIG. 4 shows the left or inner side; and

FIG. 5 is a schematic representation of a cross-section taken along line V-V of FIG. 1, to an enlarged scale.

Throughout the drawings, like integers are referenced by the same numeral and, throughout the specification, the adjective “orthotic” is used to qualify. “device”, instead of the alternative “orthodic”, “orthopedic” or “orthopaedic”.

### BEST MODE FOR CARRYING OUT THE INVENTION

The drawings illustrate a moldable, pre-made orthotic device, generally referenced 1, and comprising, inter-alia, a main part 2 which may well be of such a material as ethyl vinyl acetate (E.V.A.) having a density in the range 150-350 kg/M³ durometer. The specifications of ‘top-of-the-range’ E.V.A. and the preferred 220 kg/M³ E.V.A. are as in the following table:

| PROPERTY & TEST METHOD | UNITS | “E.V.A.” | “E.V.A.” |
|---|---|---|---|
| DENSITY | Kg/M³ | 350 | 220 |
| HARDNESS (jis type C) | — | 74 | 57 |
| WATER ABSORPTION | gms/cm³ | <0.002 | <0.002 |
| (jis K6767) | | | |
| THERMAL CONDUCTIVITY | W/M° C. | 0.064 | 0.055 |
| (astm 578) | | | |
| OPERATING TEMPERATURE | Min ° C. | −70° C. | −70° C. |
| | Max ° C. | 70° C. | 70° C. |
| TENSILE STRENGTH | MPa | 3.0 | 2.0 |
| (jis K6767) | | | |
| TEAR STRENGTH | N/Cm | 170 | 120 |
| (jis K6767) | | | |
| ELONGATION AT BREAK | % | 250-300 | 250-300 |
| (jis K6767) | | | |
| COMPRESSION SET | % | <5 | <5 |
| COMPRESSION DEFLECTION | KPa | 950 | 240 |
| (astm d3575) | | | |

The orthotic device according to the invention is manufactured on a template cast to fit a variety of foot/shoe shapes and sizes. Sizes particularly envisaged are: childrens' sizes; extra small; small, medium; large and extra large, all in regular and wide fittings.

Each orthotic device 1, is formed from a positive cast based on that same configuration as known rigid or thoses usually are. The length of the device is ideally. three-quarters of the length of the inside of the article of footwear in which it is to be placed, so as to underlie the heel bone, or os calcis, those bones between the phalangeal bones and the seven tarsal bones that articulate the foot.

Each orthotic device 1 is formed with an integrally-molded heel cup 3, a longitudinal arch ‘raise’ 4 and a metatarsal ‘raise’ 5 for the purpose of aligning the heads of the second, third and fourth metatarsals. A 4°-angled (or thereabouts) rear foot post—that is to say, a so-called ‘varus post’—is in-built to allow for leg curvature and to prevent excess pronation.

The main part 2 of orthotic device 1 is formed with a recess in the integral heel cup 3 to receive a low-density sponge-like, shock-absorbing insert 6 which is adapted to underlie that area of a patient's foot directly beneath the heel bone or os calcis, so as to cushion the foot throughout its ‘heel strike’—in other words, to absorb shock during the full heel contact phase and to be functional therethrough.

It is noteworthy that the prior art devices tend to provide cushioning at the middle and ends only of heel contact area. Advantageously, shock-absorbing insert 6 is formed from polyurethane foam material having a density in the range 30-50 kg/M³.

The shock-absorbing insert 6 of each orthotic device in accordance with the present invention ideally includes an integral portion 7 which is adapted to cushion that part of a patient's foot which lies immediately beneath a heel spur, or calcaneus, thereof when such a condition is present. Such a portion 7 may appositely be termed a “shock dot”, or “shock spot”. The cushioned area may extend either fully or partially through the full thickness of the orthotic device. Advantageously, the upper surface of the inventive orthotic device may be covered, or sheathed, with a fabric-like outer “skin”—as referenced 8 in FIG. 5.

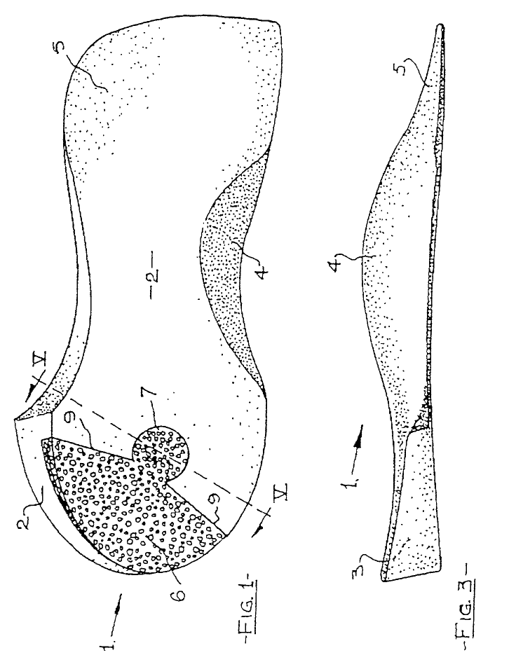

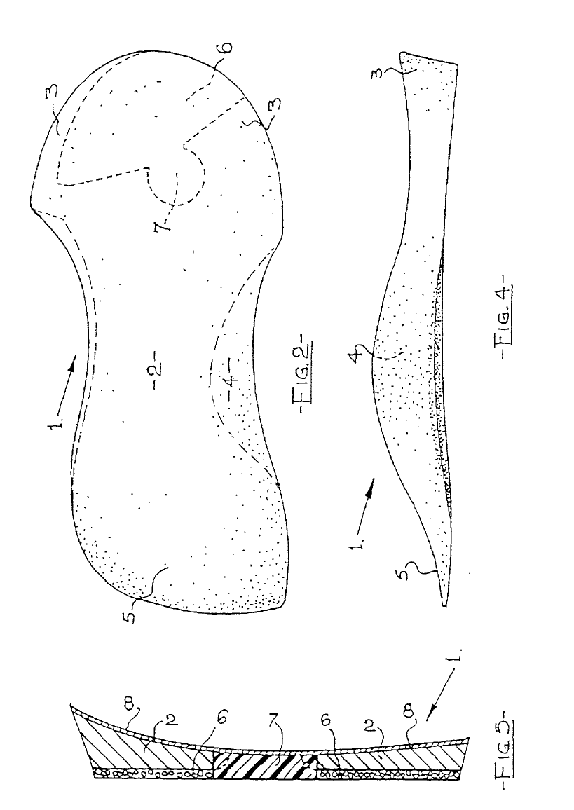

It should be noted that FIG. 5 is schematic in that it shows edges 9 of insert 6 as being visible when the. device is sectioned, refer to FIG. 1.

The width of the inventive orthotic device ideally is from the lateral aspect of the fifth metatarsal bone's head to the longitudinal bisection of the first and second metatarsals. This arrangement permits the shaft of the first metatarsal bone to “plantarflex” during the propulsive phase—that is to say, during walking or running.

Main part 2 of the orthotic device of the present invention may be manufactured by press-molding ethyl vinyl acetate (E.V.A.) and shaping by the use of grinding machinery; however, it is contemplated that a long-term production process will combine in-house “cad/cam” manufacturing procedures with possible utilization of industrial molding techniques. The recess in integral heel cup 3 may then be infilled with the 30-50 kg/M³ material, to the appropriate level, to form shock-absorbing insert 6. Alternatively, E.V.A. may be injection-molded into the mold so as to fill all but the last part and then the remaining part may be filled with the 30-50 kg/M³ foam polyurethane.

The pre-molded orthotic device 1 will have a slight tendency to mold itself to contours of the wearer's foot under the influence of body heat and wear, particularly if its density is less than about 220 kg/M³.

While a substantially three-quarter length inventive orthotic device has been described and illustrated, it is nevertheless envisaged that half- and full-length devices may well be provided as necessary or desired.

Industrial Applicability

The present invention offers several distinct advantages over and above the prior art devices: greater control of the osseous structures of the foot due to the premolded configuration, with additioal control available for customising from the heat moldable properties of the E.V.A. material used; greater and more accurate rearfoot control from the 4° rearfoot varus angle or wedge, which helps to prevent over pronation during function, along with the solid high. molded heel cup, all designed for greater calcaneal support; integrated cushioning “shock dot”; correctly contoured for metatarsal alignment, longitudinal arch alignment lateral arch alignment and sagittal calcaneal alignment; greater stability due to the basic solidity of E.V.A. and to the wide surface rear foot area; in-built varus post.

Tests have shown that the inventive inexpensive orthotic device is well able to provide a remedy for common biomechanical problems relating to the foot as listed hereinafter: heel spurs; plantar fasciitis; metatarsalgia” claw toes; calcaneal apophysitis; archilles tendonitis; shin splints; excess pronation; patella tracking malfunctions; flat feet.

From the abovegoing, it will be appreciated by those skilled in the art that numerous variations and modifications may be made to the invention without materially departing from the spirit and scope thereof, as set out in the following claims.

1. A contoured moldable orthotic device adapted to be inserted into an article of footwear so as to overlie at least a part of the upper surface of a sole thereof, to thereby give support to, and to control, the osseous structures of the foot; said orthotic device having a premolded configuration but adapted to be further moldable insitu, said orthotic device being formed with an integrally-molded heel cup, a longitudinal arch raise, a varus post angled at about 4° and a metatarsal raise for aligning the heads of the second, third and fourth metatarsals; the said heel cup incorporating a low-density sponge-like, shock-absorbing insert adapted to underlie that area of a patient's foot which is beneath the heel bone, to thereby cushion the foot throughout the heel strike thereof.

2. A moldable orthotic device as claimed in claim 1, wherein said shock-absorbing insert includes an integral “shock dot” portion adapted to cushion that part of a said patient's foot which lies immediately beneath a heel spur, or calcaneus, thereof.

3. A moldable orthotic device as claimed in claim 2, wherein the said “shock dot” portion extends either partially or fully through the full thickness of the moldable orthotic device.

4. A moldable orthotic device as claimed in claim 1 or claim 2, wherein the width of said molded orthotic device is the distance between the lateral aspect of the head of the fifth metatarsal and the longitudinal bisection of the first and second metatarsal; the arrangement being such that the shaft of the first metatarsal is able to plantarflex during the propulsive phase of a said patient's foot.

5. A moldable orthotic device as claimed in claim 1 or claim 2, wherein the upper surface of said device is sheathed in a fabric-like outer skin.

6. A moldable orthotic device as claimed in claim 1 or claim 2, wherein said shock-absorbing insert is formed from polyurethane foam having a density in the range 30-50 kg/M³ the remainder of said device being formed from ethyl vinyl acetate having a density in the range 150-350 kg/M³.

7. A moldable orthotic device as claimed in claim 6, wherein the said remainder of said device is formed from ethyl vinyl acetate having a density of 220 kg/M³.

8. A moldable orthotic device as claimed in claim 1 or claim 2, wherein the width of said device is the distance from the lateral aspect of the head of the fifth metatarsal to the longitudinal bisection of the first and second metatarsals; the. arrangement being such that the shaft of the first metatarsal is able to plantarflex during the propulsive phase of a said patient's foot.

9. A moldable orthotic device as claimed in claim 1 or claim 2, wherein the outer edge of the said device is cut away laterally, to thereby improve the fitting of the device into a patient's shoe.
